(19) United States
(12) Patent Application Publication
(10) Pub. No.: US 2025/0302413 A1
(43) Pub. Date: Oct. 2, 2025

(54) AUTOMATIC SCORING METHOD AND AUTOMATIC SCORING SYSTEM FOR ABDOMINAL AORTIC CALCIFICATION

(71) Applicant: THE FIRST AFFILIATED HOSPITAL OF NINGBO UNIVERSITY, Ningbo (CN)

(72) Inventors: Yuning PAN, Ningbo (CN); Zhehao ZHANG, Ningbo (CN); Zhenhong SHAO, Ningbo (CN); Qingqing LU, Ningbo (CN); Xiaorong WANG, Ningbo (CN); Jingfeng HUANG, Ningbo (CN); Quanliang MAO, Ningbo (CN); Yangyang QIN, Ningbo (CN); Huiying WANG, Ningbo (CN); Mingrui SONG, Ningbo (CN)

(21) Appl. No.: 18/977,275

(22) Filed: Dec. 11, 2024

(30) Foreign Application Priority Data
Mar. 27, 2024 (CN) 2024103545278

(51) Int. Cl. A61B 6/50; A61B 6/00; G06T 7/00; G16H 30/40

(52) U.S. Cl. CPC A61B 6/505; A61B 6/5217; G06T 7/0012; G16H 30/40; G06T 2207/10116; G06T 2207/20081; G06T 2207/30012

(57) ABSTRACT

Disclosed are an automatic scoring method and an automatic scoring system for abdominal aortic calcification to simulate the doctor's scoring process. The CNN is first used to locate and recognize the aorta from the first lumbar vertebra to the fourth lumbar vertebra and the aorta in the corresponding area, then an improved regression model is used to automatically perform the scoring task. With particular attention to the continuity of sample data, a regression model that can capture the continuity of the intrinsic ordered relationship between samples is designed to ensure that the changing trend of the calcification degree is reflected more accurately. A regression model is constructed based on the intrinsic continuity of samples, which solves the problem of ignoring data continuity in the direct regression method, making the scoring to be more reflective of the continuous changes in calcification severity.

### TECHNICAL FIELD

The present disclosure relates to the field of artificial intelligence applications, and in particular to an automatic scoring method and an automatic scoring system for abdominal aortic calcification.

### BACKGROUND

Abdominal aortic calcification (AAC) is a common pathological phenomenon that marks the occurrence and development of atherosclerotic cardiovascular disease. The detection and evaluation of the extent and distribution of abdominal aortic calcification can help to discover and effectively manage such diseases early, thereby improving the prevention and treatment of cardiovascular diseases. Due to low cost and high reliability, lateral lumbar spine X-rays are often used to assess the extent and range of AAC lesions in clinical practice, especially the calcification of the anterior and posterior walls of the abdominal aorta along the first lumbar vertebra to the fourth lumbar vertebra.

The 24 point semi-quantitative scoring method developed by Kauppila is a standard method specifically used to evaluate abdominal aortic calcification lesions in this specific area. According to the proportion of aortic wall area occupied by calcification (1 point for less than or equal to ⅓, 2 points for greater than ⅓ to less than or equal to ⅔, and 3 points for greater than ⅔), the anterior wall and the posterior wall are scored separately, and then the scores are added together; the total score can reach up to 24 points. However, this scoring method based on manual observation and subjective judgment relies on the doctor's experiences and has a certain degree of subjectivity, so it is difficult to ensure accuracy and consistency.

In view of this, there is an urgent need to develop a system or method that is capable of automatically detecting and quantifying the scores of abdominal aortic calcification.

### SUMMARY

In order to reduce the dependence on subjective judgment based on the doctor's experiences and to achieve automated assessment of abdominal aortic calcification, the present disclosure provides an automatic scoring method for abdominal aortic calcification, including the steps of:
S1: acquiring a plurality of lateral abdominal X-ray images of the area where the first lumbar vertebra to the fourth lumbar vertebra are located and the abdominal aorta calcification scores are known;
S2: acquiring vertebral center point heatmap and vertebral endpoint offset results from lateral abdominal X-ray images with known calcification scores by using a trained and validated vertebral localization network;
S3: acquiring central prediction points by means of maximum pooling and non-maximum suppression of the vertebral center point heatmap, and acquiring vertebral key points according to the vertebral endpoint offset results on the basis of the center prediction points;
S4: acquiring the bilateral midpoints between two vertebrae according to the key points of each vertebra, and acquiring the regional image blocks of the abdominal aorta of each lumbar vertebra by equidistantly extending the distance between the bilateral midpoints;
S5: training an ACC automatic scoring network based on Learning to Rank under the constraints of regression loss and ranking loss according to the regional image blocks of each lumbar abdominal aorta;
S6: performing abdominal aortic calcification scoring by means of a trained ACC automatic scoring network.

Further, in the step S1, the lateral abdominal X-ray images are acquired under the same lateral state and preprocessed to unify the size of the lateral abdominal X-ray images.

Further, in the step S2, the training and validation of the vertebral localization network include the following steps:
collecting the lateral abdominal X-ray images of the first lumbar vertebra to the fourth lumbar vertebrae and marking the four vertebral endpoints as an image dataset;
training and validating the vertebral localization network by extracting the image features in the image dataset and acquiring the results of the vertebral endpoint offset after obtaining the vertebral center point heatmap.

Further, in the step S2, the image dataset is expanded by the Gaussian heatmap, and the formula is expressed as follows:

$$H ( X , Y ) = exp ( - \frac{{( X - {X}_{N} )}^{2} + {( Y - {Y}_{N} )}^{2}}{2 {σ}^{2}} )$$

where, X and Y are horizontal and vertical coordinates of the target center point after expansion, respectively, X_{N} and Y_{N} are coordinates of the center point, respectively, and σ is used to control the value of the Gaussian heatmap size.

Further, in the step S2, the training of the vertebral localization network is constrained by the center point heatmap loss and the vertebral endpoint offset loss, wherein the formula for the center point heatmap loss is expressed as follows:

$${L}_{hm} = - \frac{1}{N} {\sum }_{i = 1} \{ \begin{matrix}{( 1 - {p}_{i} )}^{α} log {p}_{i} & {y}_{i} = 0 \\ {( 1 - {y}_{i} )}^{β} {( {p}_{i} )}^{α} log ( 1 - {p}_{i} ) & otherwise\end{matrix}$$

where Lₕₘ is the center point heatmap loss, i represents the i-th sample among N samples, and pᵢ and yᵢ represent the predicted value and the label at the i-th position, respectively; α and β are the coefficients used to control the loss size for hard-to-classify samples and to increase the loss for samples close to positive samples, respectively;
the vertebral endpoint offset loss is expressed as follows:

$${L}_{coroff} = \frac{1}{N} {\sum }_{i = 1}^{N} {‖ {C}^{′} - C ‖}_{2}$$

where L_{coroff} is the vertebral endpoint offset loss, i represents the i-th sample among N samples, C represents the label, and C′ represents the predicted result.

Further, after the step S2, the method further includes the step of:
unifying the size of center point labels and the model outputs by scaling the center point heatmap and the vertebral endpoints at a preset ratio.

Further, in the step S3, the vertebral key points include the left upper endpoint, left lower endpoint, right upper endpoint, and right lower endpoint of the vertebra.

Further, in the step S4, the bilateral midpoints between two vertebrae are acquired in the following manner:
the left lower endpoint or the right lower endpoint of the current vertebra are taken as edge points, the left upper endpoint or the right upper endpoint of the next adjacent vertebra are taken as edge points, and the center point of the two edge points on the same side is taken as a left midpoint or a right midpoint.

Further, in the step S4, the regional image block is acquired in the following manner:
if the lumbar vertebra is the first lumbar vertebra or the fourth lumbar vertebra,
taking the distance between the left upper endpoint and the right upper endpoint of the first lumbar vertebra as an extension length, based on the connecting line between the left upper endpoint and the right upper endpoint of the first lumbar vertebra, the edge connecting line under the extension of the vertebra in the reverse bow direction is acquired;
taking the distance between the left lower endpoint and the right lower endpoint of the fourth lumbar vertebra as an extension length, and based on the connecting line between the left lower endpoint and the right lower endpoint of the fourth lumbar vertebra, the edge connecting line under the extension of the vertebra in the reverse bow direction is acquired;
the midpoint between the lower left endpoint of the first lumbar vertebra and the upper left endpoint of the second lumbar vertebra is the left midpoint, and the midpoint between the lower right endpoint of the first lumbar vertebra and the upper right end point of the second lumbar vertebra is the right midpoint; taking the distance between bilateral midpoints of two vertebrae as an extension length, and based on the connecting line between bilateral midpoints of two vertebrae, the median connecting line under the extension of the vertebra in the reverse bow direction is acquired;
the midpoint between the left lower end point of the third lumbar vertebra and the left upper end point of the fourth lumbar vertebra is taken as the left midpoint, the midpoint between the right lower end point of the third lumbar vertebra and the right upper end point of the fourth lumbar vertebra is taken as the right midpoint, the distance between the bilateral midpoints of the two vertebrae is taken as the extension length, taking the distance between bilateral midpoints of two vertebrae as an extension length, and based on the connecting line between bilateral midpoints of two vertebrae, the median connecting line under the extension of the vertebra in the reverse bow direction is acquired;
the area surrounded by the edge connecting line and the adjacent median connecting line is a target area image block;
if the lumbar vertebra is the second lumbar vertebra or the third lumbar vertebra,
taking the distance between bilateral midpoints of two adjacent vertebrae as an extension length, and based on the connecting line between bilateral midpoints of two adjacent vertebrae, the median connecting line under the extension of the vertebra in the reverse bow direction is acquired;
the area surrounded by the median connecting line and the adjacent median connecting line is a target area image block.

The present disclosure further provides an automatic scoring system for abdominal aortic calcification, including:
a data acquisition module, configured to acquire a plurality of lateral abdominal X-ray images of the area where the first lumbar vertebra to the fourth lumbar vertebra are located and the abdominal aorta calcification scores are known;
a point acquisition module, configured to acquire vertebral center point heatmap and vertebral endpoint offset results from lateral abdominal X-ray images with known calcification scores by using a trained and validated vertebral localization network, and acquire central prediction points by means of maximum pooling and non-maximum suppression of the vertebral center point heatmap, and acquire vertebral key points according to the vertebral endpoint offset results on the basis of the center prediction points;
an image block acquisition module, configured to acquire the bilateral midpoints between two vertebrae according to the key points of each vertebra, and to acquire the regional image blocks of the abdominal aorta of each lumbar vertebra by equidistantly extending the distance between the bilateral midpoints;
a model training module, configured to train an ACC automatic scoring network based on Learning to Rank under the constraints of regression loss and ranking loss according to the regional image blocks of each lumbar abdominal aorta;
an automatic scoring module, configured to perform abdominal aortic calcification scoring by means of a trained ACC automatic scoring network.

Further, in the data acquisition module, the lateral abdominal X-ray images are acquired under the same lateral state and preprocessed to unify the size of the lateral abdominal X-ray images.
training and validating the vertebral localization network by extracting the image features in the image dataset and acquiring the results of the vertebral endpoint offset after obtaining the vertebral center point heatmap.

Further, in the point acquisition module, the image dataset is expanded by the Gaussian heatmap, and the formula is expressed as follows:

$$H ( X , Y ) = exp ( - \frac{{( X - {X}_{N} )}^{2} + {( Y - {Y}_{N} )}^{2}}{2 {σ}^{2}} )$$

where, X and Y are horizontal and vertical coordinates of the target center point after expansion, respectively, X_{N} and Y_{N} are coordinates of the center point, respectively, and σ is used to control the value of the Gaussian heatmap size.

Further, in the point acquisition module, the training of the vertebral localization network is constrained by the center point heatmap loss and the vertebral endpoint offset loss, wherein the formula for the center point heatmap loss is expressed as follows:

$${L}_{h m} = - \frac{1}{N} {\sum }_{i = 1} \{ \begin{matrix}{( 1 - {p}_{i} )}^{α} log {p}_{i} & {y}_{i} = 0 \\ {( 1 - {y}_{i} )}^{β} {( {p}_{i} )}^{α} log ( 1 - {p}_{i} ) & otherwise\end{matrix}$$

where Lₕₘ is the center point heatmap loss, i represents the i-th sample among N samples, and pᵢ and yᵢ represent the predicted value and the label at the i-th position, respectively; α and β are the coefficients used to control the loss size for hard-to-classify samples and to increase the loss for samples close to positive samples, respectively;
the vertebral endpoint offset loss is expressed as follows:

$${L}_{coroff} = \frac{1}{N} {\sum }_{i = 1}^{N} {‖ {C}^{′} - C ‖}_{2}$$

where L_{coroff} is the vertebral endpoint offset loss, i represents the i-th sample among N samples, C represents the label, and C′ represents the predicted result.

Further, in the point acquisition module, the center point labels and the model outputs are unified in size by scaling the center point heatmap and the vertebral endpoints at a preset ratio.

Further, in the point acquisition module, the vertebral key points include the left upper endpoint, left lower endpoint, right upper endpoint, and right lower endpoint of the vertebra.

Further, in the image block acquisition module, the bilateral midpoints between two vertebrae are acquired in the following manner:
the left lower endpoint or the right lower endpoint of the current vertebra are taken as edge points, the left upper endpoint or the right upper endpoint of the next adjacent vertebra are taken as edge points, and the center point of the two edge points on the same side is taken as a left midpoint or a right midpoint.

Further, in the image block acquisition module, the regional image block is acquired in the following manner:
if the lumbar vertebra is the first lumbar vertebra or the fourth lumbar vertebra,
taking the distance between the left upper endpoint and the right upper endpoint of the first lumbar vertebra as an extension length, based on the connecting line between the left upper endpoint and the right upper endpoint of the first lumbar vertebra, the edge connecting line under the extension of the vertebra in the reverse bow direction is acquired;
taking the distance between the left lower endpoint and the right lower endpoint of the fourth lumbar vertebra as an extension length, and based on the connecting line between the left lower endpoint and the right lower endpoint of the fourth lumbar vertebra, the edge connecting line under the extension of the vertebra in the reverse bow direction is acquired; the midpoint between the lower left endpoint of the first lumbar vertebra and the upper left endpoint of the second lumbar vertebra is the left midpoint, and the midpoint between the lower right endpoint of the first lumbar vertebra and the upper right end point of the second lumbar vertebra is the right midpoint; taking the distance between bilateral midpoints of two vertebrae as an extension length, and based on the connecting line between bilateral midpoints of two vertebrae, the median connecting line under the extension of the vertebra in the reverse bow direction is acquired;
the midpoint between the left lower end point of the third lumbar vertebra and the left upper end point of the fourth lumbar vertebra is taken as the left midpoint, the midpoint between the right lower end point of the third lumbar vertebra and the right upper end point of the fourth lumbar vertebra is taken as the right midpoint, the distance between the bilateral midpoints of the two vertebrae is taken as the extension length, taking the distance between bilateral midpoints of two vertebrae as an extension length, and based on the connecting line between bilateral midpoints of two vertebrae, the median connecting line under the extension of the vertebra in the reverse bow direction is acquired;
the area surrounded by the edge connecting line and the adjacent median connecting line is a target area image block;
if the lumbar vertebra is the second lumbar vertebra or the third lumbar vertebra,
taking the distance between bilateral midpoints of two adjacent vertebrae as an extension length, and based on the connecting line between bilateral midpoints of two adjacent vertebrae, the median connecting line under the extension of the vertebra in the reverse bow direction is acquired;
the area surrounded by the median connecting line and the adjacent median connecting line is a target area image block.

Compared with the prior art, the present disclosure has at least the following beneficial effects:
(1) The automatic scoring method and the automatic scoring system for abdominal aortic calcification provided in the present disclosure realizes automatic scoring by means of convolutional neural network, substitutes the traditional scoring methods that rely on the doctor's subjective experiences, thereby reducing the scoring subjectivity and improving the reliability and consistency of scoring;
(2) A regression model is constructed by means of Learning to Rank method based on the intrinsic continuity of samples, which solves the problem of ignoring data continuity in the direct regression method, making the scoring to be more reflective of the continuous changes in calcification severity;
(3) By referring to the doctor's abdominal aortic calcification scoring method, the abdominal aorta region where a single vertebra is located is evaluated and integrated separately, which is conducive to a comprehensive understanding of the patient's abdominal aortic sclerosis condition.

### BRIEF DESCRIPTION OF THE DRAWINGS

FIG. 1 is a diagram depicting steps for an automatic scoring method for abdominal aortic calcification;

FIG. 2 is a diagram depicting modules for an automatic scoring system for abdominal aortic calcification;

FIG. 3 is a schematic diagram for acquiring a regional image block.

### DETAILED DESCRIPTION OF THE EMBODIMENTS

The technical solutions of the present disclosure are further described in conjunction with the specific embodiments and accompanying drawings, but the present disclosure is not limited to these embodiments.

### Example 1

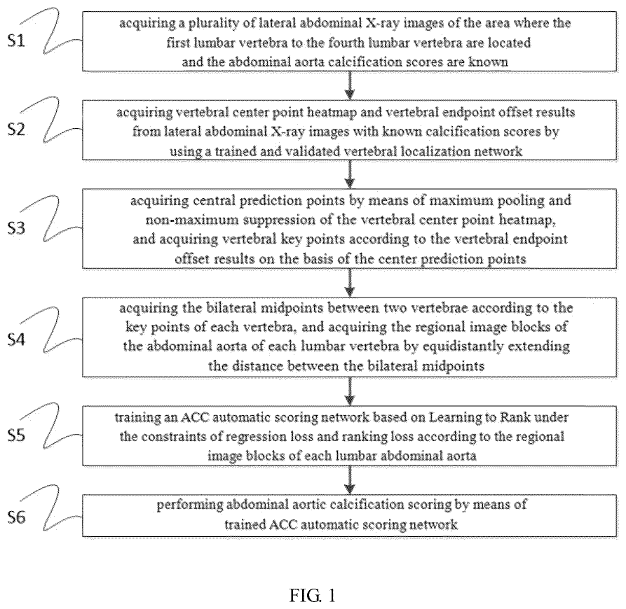

Convolutional neural network (CNN) has been widely used in medical image-assisted diagnosis due to its powerful image feature extraction ability. Based on the CNN technology, the doctor's scoring process can be imitated and a CNN model can be constructed to recognize the aorta from the first lumbar vertebra to the fourth lumbar vertebra and the area where they are located, and then the aforementioned automatic scoring is achieved by means of a regression model. However, the direct regression method inevitably ignores the continuity of data, leading to a suboptimal representation of the regression task. Therefore, in order to improve the model's attention to the intrinsic continuity of samples, the present disclosure provides an automatic scoring method for abdominal aortic calcification, which ranks the distances of features in the embedding space by referring to the intrinsic continuity of the sample AAC scores to capture the continuous representation of the intrinsic ordered relationship. As shown in FIG. 1, the method specifically includes the steps of:
S1: acquiring a plurality of lateral abdominal X-ray images of the area where the first lumbar vertebra to the fourth lumbar vertebra are located and the abdominal aorta calcification scores are known;
S2: acquiring vertebral center point heatmap and vertebral endpoint offset results from lateral abdominal X-ray images with known calcification scores by using a trained and validated vertebral localization network;
S3: acquiring central prediction points by means of maximum pooling and non-maximum suppression of the vertebral center point heatmap, and acquiring vertebral key points according to the vertebral endpoint offset results on the basis of the center prediction points;
S4: acquiring the bilateral midpoints between two vertebrae according to the key points of each vertebra, and acquiring the regional image blocks of the abdominal aorta of each lumbar vertebra by equidistantly extending the distance between the bilateral midpoints;
S5: training an ACC automatic scoring network based on Learning to Rank under the constraints of regression loss and ranking loss according to the regional image blocks of each lumbar abdominal aorta;
S6: performing abdominal aortic calcification scoring by means of a trained ACC automatic scoring network.

Wherein, the vertebral localization network includes a UNet for feature extraction, a vertebral center point prediction module and a vertebral endpoint offset prediction module. The vertebral center point prediction module includes a convolution with a convolution kernel size of 3×3 and a convolution with a convolution kernel size of 1×1. The vertebral endpoint offset prediction module includes a convolution with a convolution kernel size of 3×3 and a convolution with a convolution kernel size of 7×7.

In order to enable the vertebral localization network to meet the accurate recognition of the target area in the vertebrae, N left lateral abdominal X-ray images with four marked vertebral endpoints from the first lumbar vertebra to the fourth lumbar vertebra need to be acquired, then all preprocessed images are unified to a size of 1024×512 to form a sample set (including train set and validation set). Then, the image features are extracted by UNet to obtain N feature maps with 64 channels and a size of 256×128. Then the feature maps are passed through the vertebral center point prediction module to obtain N vertebral center point heatmap with a size of 256×128, and meanwhile, the feature maps are passed through the vertebral endpoint offset module to obtain N vertebral endpoint offset results with 8 channels and a size of 256×128.

Acquisition of center point labels: In order to maintain the consistency of the label and the model output size, the coordinates of the original image and the four endpoints of the vertebra are scaled down by 4 times simultaneously. The vertebral center point is obtained by calculating the four endpoints of the vertebra. In the target result with a size of 256×128, the vertebral center is set to 1 and other positions are set to 0. Additionally, in order to increase the number of positive samples, Gaussian heatmaps are used as prediction targets:

$$H ( X , Y ) = exp ( - \frac{{( X - {X}_{N} )}^{2} + {( Y - {Y}_{N} )}^{2}}{2 {σ}^{2}} )$$

where, X and Y are horizontal and vertical coordinates of the target center point after expansion, respectively, X_{N} and Y_{N} are coordinates of the center point, respectively; σ is used to control the value of the Gaussian heatmap size, which is 1 in this embodiment.

Acquisition of vertebral offset point labels: The vertebral endpoint offset is the distance from the vertebral center point to the horizontal and vertical coordinates of the four endpoints. The distances of horizontal and vertical coordinates of each endpoint are reflected in one channel respectively, with a total of 8 channels. The final label size is 256×128×8. During the training process, the model loss includes the center point heatmap loss and the vertebral endpoint offset loss. The center point heatmap loss is expressed as follows:

$${L}_{h m} = - \frac{1}{N} {\sum }_{i = 1} \{ \begin{matrix}{( 1 - {p}_{i} )}^{a} log {p}_{i} & {y}_{i} = 0 \\ {( 1 - {y}_{i} )}^{β} {( {p}_{i} )}^{a} log ( 1 - {p}_{i} ) & otherwise\end{matrix}$$

where Lₕₘ is the center point heatmap loss, i represents the i-th sample among N samples, and pᵢ and yᵢ represent the predicted value and the label at the i-th position, respectively; α and β are the coefficients used to control the loss size for hard-to-classify samples and to increase the loss for samples close to positive samples, respectively.

The vertebral endpoint offset loss is expressed as follows:

$${L}_{coroff} = \frac{1}{N} {\sum }_{i = 1}^{N} {‖ {C}^{′} - C ‖}_{2}$$

where L_{coroff} is the vertebral endpoint offset loss, i represents the i-th sample among N samples, C represents the label, and C′ represents the predicted result.

N left lateral abdominal X-ray images are randomly selected from the train set and input into the vertebral localization network, and during the training process, the total loss function is calculated in real time. According to the calculated value of the total loss function, the model parameters are reversely updated using the gradient descent method until the number of model parameter updates is greater than the preset value, thereby obtaining a plurality of vertebral localization network to be validated. Then the X-ray images in the validation set are input into each vertebral localization network to be validated, and the calculated total loss function values corresponding to each model to be validated are acquired. The network to be validated with the smallest calculated total loss function value is the vertebral localization network.

Then, by using the trained and validated vertebral localization network, the vertebral center point heatmap with a size of 256×128 and 8 channels and the vertebral endpoint offset results with a size of 256×128 from lateral abdominal X-ray images with known calcification scores are acquired. By means of maximum pooling with a size of 3×3 and non-maximum suppression of the vertebral center point heatmap, the largest 4 points are selected as the center prediction points of the first lumbar vertebra to the fourth lumbar vertebra. Then, based on the central prediction points, the four key points of each vertebra (i.e., the left upper endpoint, left lower endpoint, right upper endpoint, and right lower endpoint of the vertebra) are calculated separately according to the vertebral endpoint offset results, with a total of 16 key points, denoted as S_{j,k}, where j represents the j-th vertebra, k∈{tl,tr,bl,br}, is a set consisting of the left upper endpoint, left lower endpoint, right upper endpoint, and right lower endpoint of the vertebra.

According to the key points acquired, the midpoints between two vertebrae from the first lumbar vertebra to the fourth lumbar vertebra are calculated, denoted as C_{i,i+1,t}, which represents the left endpoints or the right endpoints between the i-th vertebra and the (i+1)-th vertebra. Where t∈{cl,cr}, represents the left midpoint and the right midpoint between vertebrae. For example, C_{1,2,cl}=(S_{1,bl}+S_{2,tl})/2 represents the left endpoint between the first vertebra and the second vertebra, which is obtained by calculating the coordinate average of the left lower endpoint of the first vertebra and the left upper endpoint of the second vertebra.

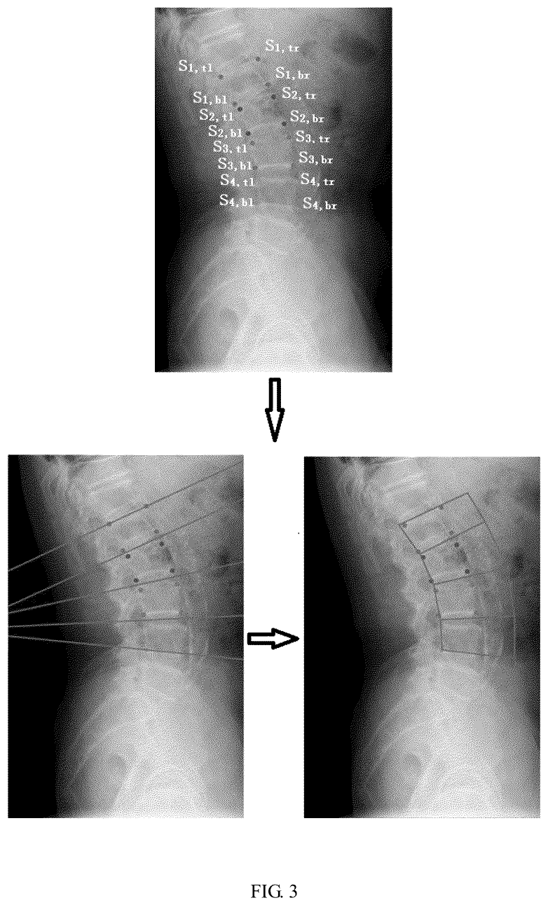

As shown in FIG. 3, S_{1,tl} and S_{1,tr} are connected and extended, and an extension point with a distance from S_{1,tr} equal to the distance between S_{1,tl} and S_{1,tr} is taken; C_{1,2,cl} and C_{1,2,cr} are connected, and an extension point with a distance from C_{1,2,cr} equal to the distance between C_{1,2,cl} and C_{1,2,cr} is taken. The above two points are connected with C_{1,2,cl} and S_{1,tl} to form a quadrangle, as the area where the abdominal aorta of the first lumbar vertebra is located. Similarly, the area where the abdominal aorta of the first lumbar vertebra to the fourth lumbar vertebra is located can be obtained in a left lateral abdominal radiograph according to the above method.

The ACC automatic scoring network is trained based on the above obtained image blocks of the lumbar abdominal aorta region. Here, the AAC automatic scoring network extracts features through ResNet50 and adopts a regression method to evaluate AAC after the features are passed through a fully connected layer. The output of ResNet50 is a 512-dimensional feature vector F, which is passed through a fully connected layer to obtain a 128-dimensional feature vector, and then passed through a fully connected layer to obtain a 1-dimensional vector y to represent the AAC score.

First, a certain number of left lateral abdominal X-ray images with known AAC scores of the abdominal aorta region where the four vertebrae from the first lumbar vertebra to the fourth lumbar vertebra are located are obtained. Then all preprocessed images are normalized to a uniform size of 1024×512. The image blocks of the abdominal aorta region where the four vertebral key points are located are obtained by using the center point heatmap and the vertebral endpoint offset results in the previous way, to form a sample set.

N image blocks are selected from the sample set and unified to a size of 512×256 by bilinear interpolation and input into the constructed AAC automatic scoring network. For the training of the AAC automatic scoring network, the AAC regression loss of the network is:

L_{reg}=1/NΣᵢ₌₁^{4N}∥∥y′−y∥₂

Where L_{reg} is the regression loss, y is the ACC score predicted by the model, and y′ represents the AAC score label of the input network sample. For samples vi, vj and vk, d(·,·) represents the L₁ distance between the corresponding labels of the two samples. If S_{i,j}={F_{R}|k≠i, d(vᵢ, vₖ)≥d(vᵢ, vⱼ)} is met (where S_{i,j} is a set of conditions that meet the requirements and Fₖ is a feature), then d(Fᵢ, Fₖ) is ≥d(Fᵢ, Fⱼ). Based on this, the ranking loss of the network is:

$${L}_{C R} = \frac{1}{N} {\sum }_{i = 1}^{N} \frac{1}{N - 1} {\sum }_{j = 1 , j \neq i}^{4} - log \frac{exp ( sim ( {F}_{i} , {F}_{j} ) / τ )}{{\sum }_{{F}_{k} ∈ {S}_{i , j}} exp ( sim ( {F}_{i} , {F}_{k} ) / τ )}$$

Where τ is the temperature coefficient,

$$sim ( a , b ) = \frac{a \cdot b}{\vert a \vert \cdot \vert b \vert}$$

represents the similarity of the two feature vectors a and b.

The comparison and ranking is performed according to the AAC scores of N samples to optimize the distance between samples. Multiple AAC automatic scoring networks to be validated are obtained by repeatedly training the vertebral localization network through a train set, and the total loss function is calculated in real time during the training process. The total loss is the sum of the regression loss and the ranking loss. According to the calculated value of the total loss function, the model parameters are reversely updated using the gradient descent method until the number of model parameter updates is less than the preset value.

Finally, the X-ray images in the validation set are input into each AAC automatic scoring network to be validated, and the calculated total loss function values corresponding to each model to be validated are acquired. The network to be validated with the smallest calculated total loss function value is acquired as the AAC automatic scoring network. The sum of the ACC scores of the abdominal aorta region where the first lumbar vertebra to the fourth lumbar vertebra are located in the same image is the ACC score of the entire image.

### Example 2

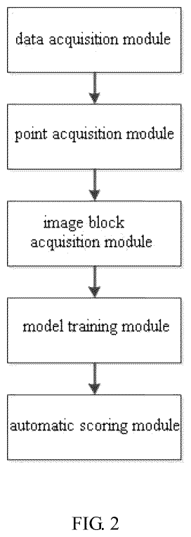

In order to facilitate understanding of the technical content of the present disclosure, the present disclosure is described in the form of a system structure in this embodiment. As shown in FIG. 2, an automatic scoring system for abdominal aortic calcification includes:
a data acquisition module, configured to acquire a plurality of lateral abdominal X-ray images of the area where the first lumbar vertebra to the fourth lumbar vertebra are located and the abdominal aorta calcification scores are known;
a point acquisition module, configured to acquire vertebral center point heatmap and vertebral endpoint offset results from lateral abdominal X-ray images with known calcification scores by using a trained and validated vertebral localization network, and acquire central prediction points by means of maximum pooling and non-maximum suppression of the vertebral center point heatmap, and acquire vertebral key points according to the vertebral endpoint offset results on the basis of the center prediction points;
an image block acquisition module, configured to acquire the bilateral midpoints between two vertebrae according to the key points of each vertebra, and to acquire the regional image blocks of the abdominal aorta of each lumbar vertebra by equidistantly extending the distance between the bilateral midpoints;
a model training module, configured to train an ACC automatic scoring network based on Learning to Rank under the constraints of regression loss and ranking loss according to the regional image blocks of each lumbar abdominal aorta;
an automatic scoring module, configured to perform abdominal aortic calcification scoring by means of a trained ACC automatic scoring network.

Further, in the point acquisition module, the training and validation of the vertebral localization network include the following steps:
collecting the lateral abdominal X-ray images of the first lumbar vertebra to the fourth lumbar vertebrae and marking the four vertebral endpoints as an image dataset;
training and validating the vertebral localization network by extracting the image features in the image dataset and acquiring the results of the vertebral endpoint offset after obtaining the vertebral center point heatmap.

Further, in the point acquisition module, the vertebral key points include the left upper endpoint, left lower endpoint, right upper endpoint, and right lower endpoint of the vertebra.

Further, in the image block acquisition module, the bilateral midpoints between two vertebrae are acquired in the following manner:
the left lower endpoint or the right lower endpoint of the current vertebra are taken as edge points, the left upper endpoint or the right upper endpoint of the next adjacent vertebra are taken as edge points, and the center point of the two edge points on the same side is taken as a left midpoint or a right midpoint.

Further, in the image block acquisition module, the regional image block is acquired in the following manner:
if the lumbar vertebra is the first lumbar vertebra or the fourth lumbar vertebra,
the midpoint between the lower left endpoint of the first lumbar vertebra and the upper left endpoint of the second lumbar vertebra is the left midpoint, and the midpoint between the lower right endpoint of the first lumbar vertebra and the upper right end point of the second lumbar vertebra is the right midpoint; taking the distance between bilateral midpoints of two vertebrae as an extension length, and based on the connecting line between bilateral midpoints of two vertebrae, the median connecting line under the extension of the vertebra in the reverse bow direction is acquired;
the midpoint between the left lower end point of the third lumbar vertebra and the left upper end point of the fourth lumbar vertebra is taken as the left midpoint, the midpoint between the right lower end point of the third lumbar vertebra and the right upper end point of the fourth lumbar vertebra is taken as the right midpoint, the distance between the bilateral midpoints of the two vertebrae is taken as the extension length, taking the distance between bilateral midpoints of two vertebrae as an extension length, and based on the connecting line between bilateral midpoints of two vertebrae, the median connecting line under the extension of the vertebra in the reverse bow direction is acquired; the midpoint between the lower left endpoint of the first lumbar vertebra and the upper left endpoint of the second lumbar vertebra is the left midpoint, and the midpoint between the lower right endpoint of the first lumbar vertebra and the upper right end point of the second lumbar vertebra is the right midpoint; taking the distance between bilateral midpoints of two vertebrae as an extension length, and based on the connecting line between bilateral midpoints of two vertebrae, the median connecting line under the extension of the vertebra in the reverse bow direction is acquired;
the midpoint between the left lower end point of the third lumbar vertebra and the left upper end point of the fourth lumbar vertebra is taken as the left midpoint, the midpoint between the right lower end point of the third lumbar vertebra and the right upper end point of the fourth lumbar vertebra is taken as the right midpoint, the distance between the bilateral midpoints of the two vertebrae is taken as the extension length, taking the distance between bilateral midpoints of two vertebrae as an extension length, and based on the connecting line between bilateral midpoints of two vertebrae, the median connecting line under the extension of the vertebra in the reverse bow direction is acquired;
the area surrounded by the edge connecting line and the adjacent median connecting line is a target area image block;
if the lumbar vertebra is the second lumbar vertebra or the third lumbar vertebra,
taking the distance between bilateral midpoints of two vertebrae as an extension length, and based on the connecting line between bilateral midpoints of two vertebrae, the median connecting line under the extension of the vertebra in the reverse bow direction is acquired;
the area surrounded by the median connecting line and the adjacent median connecting line is a target area image block.

In summary, the automatic scoring method and the automatic scoring system for abdominal aortic calcification provided in the present disclosure realizes automatic scoring by means of convolutional neural network, substitutes the traditional scoring methods that rely on the doctor's subjective experiences, thereby reducing the scoring subjectivity and improving the reliability and consistency of scoring.

A regression model is constructed by means of Learning to Rank method based on the intrinsic continuity of samples, which solves the problem of ignoring data continuity in the direct regression method, making the scoring to be more reflective of the continuous changes in calcification severity.

By referring to the doctor's abdominal aortic calcification scoring method, the abdominal aorta region where a single vertebra is located is evaluated and integrated separately, which is conducive to a comprehensive understanding of the patient's abdominal aortic sclerosis condition.

It should be noted that all directional indications (such as up, down, left, right, front, back, etc.) in the embodiments of the present disclosure are only used to explain the relative position relationship, movement, etc. between the components under a specific posture (as shown in the figures). If the specific posture changes, the directional indication will also change accordingly.

Furthermore, in the present disclosure, the descriptions such as “first”, “second”, “one”, etc. are only used for descriptive purposes, and should not be construed as indicating or implying their relative importance or implicitly indicating the number of technical features indicated. Therefore, the features defined as “first” and “second” may explicitly or implicitly include at least one of the features. In the description of the present disclosure, “a/the plurality of” means at least two, for example, two, three, etc., unless otherwise clearly and specifically defined.

In the present disclosure, unless otherwise clearly specified and defined, the terms “connection/connected”, “fix/fixed”, etc. should be understood in a broad sense. For example, “fix/fixed” can be fixedly connected, detachably connected or integrated; can be mechanically connected or electrically connected; can be directly connected or indirectly connected through an intermediate medium, can be internally communicated or mutually interacted between two elements, unless otherwise clearly defined. For those skilled in the art, the specific meanings of the above terms in the present disclosure can be understood according to specific circumstances.

Furthermore, the technical solutions between various embodiments of the present disclosure can be mutually combined provided that these solutions can be achieved by a person of ordinary skill in the art. When the combination of technical solutions is contradictory or cannot be implemented, it should be considered that such a combination of technical solutions does not exist and is not within the scope of protection of the present disclosure.

What is claimed is:

1. An automatic scoring method for abdominal aortic calcification, comprising the steps of:
S1: acquiring a plurality of lateral abdominal X-ray images of the area where the first lumbar vertebra to the fourth lumbar vertebra are located and the abdominal aorta calcification scores are known;
S2: acquiring vertebral center point heatmap and vertebral endpoint offset results from lateral abdominal X-ray images with known calcification scores by using a trained and validated vertebral localization network;
S3: acquiring central prediction points by means of maximum pooling and non-maximum suppression of the vertebral center point heatmap, and acquiring vertebral key points according to the vertebral endpoint offset results on the basis of the center prediction points;
S4: acquiring the bilateral midpoints between two vertebrae according to the key points of each vertebra, and acquiring the regional image blocks of the abdominal aorta of each lumbar vertebra by equidistantly extending the distance between the bilateral midpoints;
S5: training an ACC automatic scoring network based on Learning to Rank under the constraints of regression loss and ranking loss according to the regional image blocks of each lumbar abdominal aorta;
S6: performing abdominal aortic calcification scoring by means of a trained ACC automatic scoring network.

2. The automatic scoring method for abdominal aortic calcification of claim 1, wherein in the step S1, the lateral abdominal X-ray images are acquired under the same lateral state and preprocessed to unify the size of the lateral abdominal X-ray images.

3. The automatic scoring method for abdominal aortic calcification of claim 1, wherein in the step S2, the training and validation of the vertebral localization network comprise the following steps:
collecting the lateral abdominal X-ray images of the first lumbar vertebra to the fourth lumbar vertebrae and marking the four vertebral endpoints as an image dataset;
training and validating the vertebral localization network by extracting the image features in the image dataset and acquiring the results of the vertebral endpoint offset after obtaining the vertebral center point heatmap.

4. The automatic scoring method for abdominal aortic calcification of claim 3, wherein in the step S2, the image dataset is expanded by the Gaussian heatmap, and the formula is expressed as follows:
$$H ( X , Y ) = exp ( - \frac{{( X - {X}_{N} )}^{2} + {( Y - {Y}_{N} )}^{2}}{2 {σ}^{2}} )$$
where, X and Y are horizontal and vertical coordinates of the target center point after expansion, respectively, X_{N} and Y_{N} are coordinates of the center point, respectively, and σ is used to control the value of the Gaussian heatmap size.

5. The automatic scoring method for abdominal aortic calcification of claim 3, wherein in the step S2, the training of the vertebral localization network is constrained by the center point heatmap loss and the vertebral endpoint offset loss, wherein the formula for the center point heatmap loss is expressed as follows:
$${L}_{h m} = - \frac{1}{N} {\sum }_{i = 1} \{ \begin{matrix}{( 1 - {p}_{i} )}^{α} log {p}_{i} & {y}_{i} = 0 \\ {( 1 - {y}_{i} )}^{β} {( {p}_{i} )}^{α} log ( 1 - {p}_{i} ) & otherwise\end{matrix}$$
where Lₕₘ is the center point heatmap loss, i represents the i-th sample among N samples, and pᵢ and yᵢ represent the predicted value and the label at the i-th position, respectively; α and β are the coefficients used to control the loss size for hard-to-classify samples and to increase the loss for samples close to positive samples, respectively;
the vertebral endpoint offset loss is expressed as follows:
$${L}_{coroff} = \frac{1}{N} {\sum }_{i = 1}^{N} {‖ {C}^{′} - C ‖}_{2}$$
where L_{coroff} is the vertebral endpoint offset loss, i represents the i-th sample among N samples, C represents the label, and C′ represents the predicted result.

6. The automatic scoring method for abdominal aortic calcification of claim 1, wherein after the step S2, the method further comprises the step of:
unifying the size of center point labels and the model outputs by scaling the center point heatmap and the vertebral endpoints at a preset ratio.

7. The automatic scoring method for abdominal aortic calcification of claim 1, wherein in the step S3, the vertebral key points comprise the left upper endpoint, left lower endpoint, right upper endpoint, and right lower endpoint of the vertebra.

8. The automatic scoring method for abdominal aortic calcification of claim 7, wherein in the step S4, the bilateral midpoints between two vertebrae are acquired in the following manner:
the left lower endpoint or the right lower endpoint of the current vertebra are taken as edge points, the left upper endpoint or the right upper endpoint of the next adjacent vertebra are taken as edge points, and the center point of the two edge points on the same side is taken as a left midpoint or a right midpoint.

9. The automatic scoring method for abdominal aortic calcification of claim 8, wherein in the step S4, the regional image block is acquired in the following manner:
if the lumbar vertebra is the first lumbar vertebra or the fourth lumbar vertebra,
taking the distance between the left upper endpoint and the right upper endpoint of the first lumbar vertebra as an extension length, based on the connecting line between the left upper endpoint and the right upper endpoint of the first lumbar vertebra, the edge connecting line under the extension of the vertebra in the reverse bow direction is acquired;
taking the distance between the left lower endpoint and the right lower endpoint of the fourth lumbar vertebra as an extension length, and based on the connecting line between the left lower endpoint and the right lower endpoint of the fourth lumbar vertebra, the edge connecting line under the extension of the vertebra in the reverse bow direction is acquired;
the midpoint between the lower left endpoint of the first lumbar vertebra and the upper left endpoint of the second lumbar vertebra is the left midpoint, and the midpoint between the lower right endpoint of the first lumbar vertebra and the upper right end point of the second lumbar vertebra is the right midpoint; taking the distance between bilateral midpoints of two vertebrae as an extension length, and based on the connecting line between bilateral midpoints of two vertebrae, the median connecting line under the extension of the vertebra in the reverse bow direction is acquired;
the midpoint between the left lower end point of the third lumbar vertebra and the left upper end point of the fourth lumbar vertebra is taken as the left midpoint, the midpoint between the right lower end point of the third lumbar vertebra and the right upper end point of the fourth lumbar vertebra is taken as the right midpoint, the distance between the bilateral midpoints of the two vertebrae is taken as the extension length, taking the distance between bilateral midpoints of two vertebrae as an extension length, and based on the connecting line between bilateral midpoints of two vertebrae, the median connecting line under the extension of the vertebra in the reverse bow direction is acquired;
the area surrounded by the edge connecting line and the adjacent median connecting line is a target area image block;
if the lumbar vertebra is the second lumbar vertebra or the third lumbar vertebra,
taking the distance between bilateral midpoints of two adjacent vertebrae as an extension length, and based on the connecting line between bilateral midpoints of two adjacent vertebrae, the median connecting line under the extension of the vertebra in the reverse bow direction is acquired;
the area surrounded by the median connecting line and the adjacent median connecting line is a target area image block.

10. An automatic scoring system for abdominal aortic calcification, comprising:
a data acquisition module, configured to acquire a plurality of lateral abdominal X-ray images of the area where the first lumbar vertebra to the fourth lumbar vertebra are located and the abdominal aorta calcification scores are known;
a point acquisition module, configured to acquire vertebral center point heatmap and vertebral endpoint offset results from lateral abdominal X-ray images with known calcification scores by using a trained and validated vertebral localization network, and acquire central prediction points by means of maximum pooling and non-maximum suppression of the vertebral center point heatmap, and acquire vertebral key points according to the vertebral endpoint offset results on the basis of the center prediction points;
an image block acquisition module, configured to acquire the bilateral midpoints between two vertebrae according to the key points of each vertebra, and to acquire the regional image blocks of the abdominal aorta of each lumbar vertebra by equidistantly extending the distance between the bilateral midpoints;
a model training module, configured to train an ACC automatic scoring network based on Learning to Rank under the constraints of regression loss and ranking loss according to the regional image blocks of each lumbar abdominal aorta;
an automatic scoring module, configured to perform abdominal aortic calcification scoring by means of a trained ACC automatic scoring network.

11. The automatic scoring system for abdominal aortic calcification of claim 10, wherein in the data acquisition module, the lateral abdominal X-ray images are acquired under the same lateral state and preprocessed to unify the size of the lateral abdominal X-ray images.

12. The automatic scoring system for abdominal aortic calcification of claim 10, wherein in the point acquisition module, the training and validation of the vertebral localization network comprise the following steps:
collecting the lateral abdominal X-ray images of the first lumbar vertebra to the fourth lumbar vertebrae and marking the four vertebral endpoints as an image dataset;
training and validating the vertebral localization network by extracting the image features in the image dataset and acquiring the results of the vertebral endpoint offset after obtaining the vertebral center point heatmap.

13. The automatic scoring system for abdominal aortic calcification of claim 12, wherein in the point acquisition module, the image dataset is expanded by the Gaussian heatmap, and the formula is expressed as follows:
$$H ( X , Y ) = exp ( - \frac{{( X - {X}_{N} )}^{2} + {( Y - {Y}_{N} )}^{2}}{2 {σ}^{2}} )$$
where, X and Y are horizontal and vertical coordinates of the target center point after expansion, respectively, X_{N} and Y_{N} are coordinates of the center point, respectively, and σ is used to control the value of the Gaussian heatmap size.

14. The automatic scoring system for abdominal aortic calcification of claim 12, wherein in the point acquisition module, the training of the vertebral localization network is constrained by the center point heatmap loss and the vertebral endpoint offset loss, wherein the formula for the center point heatmap loss is expressed as follows:
$${L}_{h m} = - \frac{1}{N} {\sum }_{i = 1} \{ \begin{matrix}{( 1 - {p}_{i} )}^{α} log {p}_{i} & {y}_{i} = 0 \\ {( 1 - {y}_{i} )}^{β} {( {p}_{i} )}^{α} log ( 1 - {p}_{i} ) & otherwise\end{matrix}$$
where Lₕₘ is the center point heatmap loss, i represents the i-th sample among N samples, and pᵢ and yᵢ represent the predicted value and the label at the i-th position, respectively; α and β are the coefficients used to control the loss size for hard-to-classify samples and to increase the loss for samples close to positive samples, respectively;
the vertebral endpoint offset loss is expressed as follows:
$${L}_{coroff} = \frac{1}{N} {\sum }_{i = 1}^{N} {‖ {C}^{′} - C ‖}_{2}$$
where L_{coroff} is the vertebral endpoint offset loss, i represents the i-th sample among N samples, C represents the label, and C′ represents the predicted result.

15. The automatic scoring system for abdominal aortic calcification of claim 10, wherein in the point acquisition module, the center point labels and the model outputs are unified in size by scaling the center point heatmap and the vertebral endpoints at a preset ratio.

16. The automatic scoring system for abdominal aortic calcification of claim 10, wherein in the point acquisition module, the vertebral key points comprise the left upper endpoint, left lower endpoint, right upper endpoint, and right lower endpoint of the vertebra.

17. The automatic scoring system for abdominal aortic calcification of claim 16, wherein in the image block acquisition module, the bilateral midpoints between two vertebrae are acquired in the following manner:
the left lower endpoint or the right lower endpoint of the current vertebra are taken as edge points, the left upper endpoint or the right upper endpoint of the next adjacent vertebra are taken as edge points, and the center point of the two edge points on the same side is taken as a left midpoint or a right midpoint.

18. The automatic scoring system for abdominal aortic calcification of claim 17, wherein in the image block acquisition module, the regional image block is acquired in the following manner:
if the lumbar vertebra is the first lumbar vertebra or the fourth lumbar vertebra,
taking the distance between the left upper endpoint and the right upper endpoint of the first lumbar vertebra as an extension length, based on the connecting line between the left upper endpoint and the right upper endpoint of the first lumbar vertebra, the edge connecting line under the extension of the vertebra in the reverse bow direction is acquired;
taking the distance between the left lower endpoint and the right lower endpoint of the fourth lumbar vertebra as an extension length, and based on the connecting line between the left lower endpoint and the right lower endpoint of the fourth lumbar vertebra, the edge connecting line under the extension of the vertebra in the reverse bow direction is acquired; the midpoint between the lower left endpoint of the first lumbar vertebra and the upper left endpoint of the second lumbar vertebra is the left midpoint, and the midpoint between the lower right endpoint of the first lumbar vertebra and the upper right end point of the second lumbar vertebra is the right midpoint; taking the distance between bilateral midpoints of two vertebrae as an extension length, and based on the connecting line between bilateral midpoints of two vertebrae, the median connecting line under the extension of the vertebra in the reverse bow direction is acquired;
the midpoint between the left lower end point of the third lumbar vertebra and the left upper end point of the fourth lumbar vertebra is taken as the left midpoint, the midpoint between the right lower end point of the third lumbar vertebra and the right upper end point of the fourth lumbar vertebra is taken as the right midpoint, the distance between the bilateral midpoints of the two vertebrae is taken as the extension length, taking the distance between bilateral midpoints of two vertebrae as an extension length, and based on the connecting line between bilateral midpoints of two vertebrae, the median connecting line under the extension of the vertebra in the reverse bow direction is acquired;
the area surrounded by the edge connecting line and the adjacent median connecting line is a target area image block;
if the lumbar vertebra is the second lumbar vertebra or the third lumbar vertebra,
taking the distance between bilateral midpoints of two adjacent vertebrae as an extension length, and based on the connecting line between bilateral midpoints of two adjacent vertebrae, the median connecting line under the extension of the vertebra in the reverse bow direction is acquired;
the area surrounded by the median connecting line and the adjacent median connecting line is a target area image block.
